(19) United States
(12) Patent Application Publication
(10) Pub. No.: US 2026/0069809 A1
(43) Pub. Date: Mar. 12, 2026

(54) APPARATUS AND METHOD FOR MACHINE VISION GUIDED ENDOTRACHEAL INTUBATION

(71) Applicant: CENTRE HOSPITALIER UNIVERSI-TAIRE VAUDOIS, Epalinges (CH)

(72) Inventors: Andre Mercanzini, Saint-Sulpice (CH); Patrick Schoettker, Pully (CH)

(21) Appl. No.: 19/394,001

(22) Filed: Nov. 19, 2025

Related U.S. Application Data
(63) Continuation-in-part of application No. PCT/CH2024/050025, filed on May 22, 2024.
(60) Provisional application No. 63/468,545, filed on May 24, 2023.

(51) Int. Cl. A61M 16/04; A61M 16/00

(52) U.S. Cl. CPC A61M 16/0488; A61M 16/024; A61M 16/0418; A61M 2205/50

(57) ABSTRACT

A machine-vision guided robotic system comprises a guiding tube robotic mechanism with multiple concentric, telescopically extendable extension tubes and integrated imaging modules at their distal ends. The method for performing automated endotracheal intubation is directed by a controller that processes sequential images of anatomical regions, including the oral cavity, oropharynx, and trachea, to identify anatomical landmarks such as the teeth, epiglottis, uvula, and vocal folds. Using these features, the controller plans and executes precise robotic movements for staged advancement through the airway. The system may further adjust the curvature of the extension tubes to optimize trajectory and alignment. Once positioned, an endotracheal tube is advanced over the guiding mechanism into the trachea, after which the robotic components are retracted in reverse order. The invention enables accurate, image-guided airway access with minimal manual intervention, improving safety, repeatability, and success rates in airway management procedures.

### CROSS-REFERENCE DATA

This patent application is a continuation-in part of a co-pending international Patent Application No. PCT/CH2024/050025, filed on 22 May 2024 with the same title, which, in turn, claims a priority date benefit from the U.S. Provisional Patent Application No. 63/468,545 filed on 24 May 2023. Both documents are included herein in their respective entireties by reference.

### BACKGROUND

Without limiting the scope of the invention, its background is described in connection with an endotracheal intubation procedure. More particularly, the invention describes improvements to a robotic device and methods for automatically positioning an endotracheal tube in a subject's trachea.

Endotracheal intubation (ETI), also known as tracheal intubation (TI), is a critical medical procedure performed to establish and maintain a patent airway in subjects who are unable to breathe adequately on their own. It is routinely indicated for subjects under general anesthesia, or those experiencing respiratory failure, trauma, or altered consciousness due to illness or injury. The primary purpose of ETI is to prevent asphyxia or hypoxia by providing an open conduit through which oxygen and anesthetic gases can be delivered, and through which respiratory secretions can be managed.

During the procedure, an endotracheal tube (ETT) is inserted through the subject's mouth or nose, advanced past the glottic opening, and guided through the larynx and vocal cords into the trachea. Once the ETT is correctly positioned, a cuff near its distal end is inflated to form a seal between the tube and the tracheal wall, thereby preventing air leakage and aspiration of gastric contents. The proximal end of the tube is then connected to a ventilator or anesthesia circuit to ensure controlled delivery of oxygen and ventilation to the lungs.

Although tracheal intubation is a common and essential component of airway management, it remains a technically challenging and potentially hazardous procedure, particularly in emergency and critical care environments. Difficulty or failure to secure the airway remains a major cause of morbidity and mortality in anesthesia and critical care medicine. Adverse outcomes associated with failed or traumatic intubation include hypoxemia, aspiration, airway trauma, brain injury, cardiac arrest, and death. Despite decades of clinical practice and technological advancements, airway management errors continue to contribute significantly to preventable perioperative and critical care complications.

Predicting a difficult airway or challenging intubation scenario is inherently unreliable. While various preoperative assessments, such as Mallampati classification, thyromental distance, or cervical spine mobility, can provide guidance, they often fail to accurately identify subjects who will prove difficult to intubate. Consequently, even skilled clinicians may encounter unexpected difficulties during airway management.

Manual intubation is highly operator-dependent and demands a combination of visual-spatial skills, dexterity, and experience. The success of the procedure relies on the clinician's ability to visualize the laryngeal inlet, often using a laryngoscope, and accurately guide the ETT into the trachea while avoiding the esophagus. However, factors such as patient anatomy, body habitus, limited mouth opening, restricted cervical spine movement, the presence of secretions or blood, and suboptimal lighting can obscure visualization and complicate the process. In addition, external conditions such as poor ergonomics, time pressure, or patient instability in the intensive care unit (ICU) or emergency department (ED) can further compromise procedural success.

Operator performance also plays a critical role. Inadequate training, lack of recent experience, or staff fatigue can result in improper technique, multiple intubation attempts, and a higher risk of airway trauma. Even with the introduction of advanced video laryngoscopes and optical aids designed to enhance visualization of anatomical landmarks, the rate of complications has not significantly declined. These devices require specialized training, and their effectiveness diminishes in the absence of experienced operators.

Complications related to endotracheal intubation are common and well-documented. Laryngeal injuries, including mucosal inflammation, edema, vocal cord ulceration, granuloma formation, paralysis, and stenosis, are among the most frequent. Esophageal intubation, wherein the tube is inadvertently placed into the esophagus rather than the trachea, can result in ineffective ventilation, gastric insufflation, and rapid onset of severe hypoxemia. Evidence indicates that repeated failed attempts at intubation greatly increase the risk of such adverse outcomes. Studies show that subjects requiring more than two attempts experience a markedly higher incidence of hypoxemia, aspiration, and cardiac arrest. The risk of hypoxemia increases by approximately 51% with each additional esophageal intubation episode, and the likelihood of subsequent failure rises eleven-fold following an initial esophageal intubation.

These risks are further compounded in older subjects, in whom anatomical and physiological changes, such as reduced cervical spine mobility, diminished mouth opening, short thyromental distance, and poor dentition, contribute to greater difficulty in visualization and tube placement. The incidence of difficult intubation increases with age, and when combined with limited operator experience, it frequently results in multiple failed attempts, airway trauma, and hemodynamic instability.

Emergency intubations, performed outside the controlled setting of the operating room, such as in the ICU, ED, or prehospital environment, are associated with particularly high complication rates. In these settings, operators often face limited resources, variable lighting, unstable patients, and a lack of dedicated airway support teams. Consequently, the frequency of failed or delayed intubation, esophageal intubation, and hypoxic injury is significantly higher compared to elective surgical settings.

Despite technological advances, existing airway management tools remain heavily dependent on human judgment, coordination, and visual interpretation. Since the publication of the initial difficult airway management guidelines in 1993, numerous advanced airway devices have been introduced into clinical practice, including video laryngoscopes, fiberoptic bronchoscopes, and supraglottic airway devices. While these technologies have enhanced visualization and improved first-pass success rates in certain clinical settings, they remain fundamentally limited by their reliance on manual operation and their inability to adapt to the unique anatomical features of individual subjects. Each device operates as a fixed hardware system without real-time subject-specific feedback or adaptive control. Consequently, even with high-quality imaging systems, successful endotracheal intubation continues to depend heavily on operator skill, experience, and situational awareness.

There remains a clear and unmet need for systems that can provide automated, precise, and reliable placement of an endotracheal tube, while continuously verifying tracheal versus esophageal positioning in real time. A robotic system incorporating machine vision and intelligent actuation has the potential to overcome the limitations of manual intubation by providing consistent accuracy, reducing operator dependency, minimizing tissue trauma, and improving patient safety across both elective and emergency environments.

### SUMMARY

Accordingly, it is an object of the present invention to overcome these and other drawbacks of the prior art by providing a novel robotic system which utilizes machine vision to guide an automated insertion of an endotracheal tube and proper positioning thereof in the subject's trachea.

It is another object of the present invention to provide a robotic apparatus for guiding the endotracheal tube to a proper position in the trachea using a machine vision system trained using a neural network and a plurality of previously recorded intubation procedures.

It is a further object of the present invention to provide a robotic system to improve the reliability of the intubation procedure, reduce the number of intubation attempts and associated complications.

The present invention addresses long-standing limitations described above by providing a machine-vision guided robotic apparatus and associated method for automated or semi-automated endotracheal intubation. The disclosed apparatus integrates robotic actuation with advanced machine learning (ML) and artificial intelligence (AI) algorithms to interpret anatomical imagery, plan motion trajectories, and guide a multi-stage robotic mechanism through the oropharyngeal and tracheal regions with high precision and safety. The system is designed to reduce operator dependency, improve accuracy, and minimize the risk of tissue trauma or misplacement of the endotracheal tube.

In certain embodiments, the invention comprises a robotic component that includes a guiding tube robotic mechanism and an actuation module controlled by a central controller. The guiding tube mechanism comprises an external arm terminating in a first stabilizing component, which serves as a reference structure and initial imaging platform. The external arm is configured to capture images of the oral cavity using one or more imaging sensors.

A first extension tube is positioned concentrically within the external arm and is configured to extend distally therefrom under control of the actuation module. The distal end of the first extension tube carries imaging means configured to acquire images of the oropharynx. A second extension tube is positioned inside the first extension tube and extends distally to visualize the epiglottis, and a third extension tube is positioned inside the second extension tube and extends further distally to reach the tracheal lumen. Each extension stage provides a controlled, incremental advance toward the target airway while continuously updating positional information through captured imagery.

The controller operates the robotic component by performing machine-vision analysis on images captured from each stage of advancement. Using trained ML/AI algorithms, the controller identifies relevant anatomical structures such as the tongue, uvula, epiglottis, vocal cords, and tracheal rings, and calculates optimal motion paths that avoid contact with surrounding tissue. This enables adaptive trajectory planning and real-time correction of the robotic arm's position and orientation based on the subject's individual anatomy. The apparatus may also incorporate safety mechanisms such as force sensing, pressure feedback, and image-based validation to prevent excessive tissue pressure or misdirection of the endotracheal tube.

In one embodiment, the method of machine-vision guided endotracheal intubation using the disclosed apparatus includes the following key steps:
(a) preparation and positioning of an endotracheal tube over the first extension tube of the robotic guiding mechanism,
(b) placement of the stabilizing component of the external arm within the oral cavity adjacent to a positional marker or reference structure, such as the teeth or hard palate,
(c) acquisition and processing of images of the oral cavity to determine the position of the stabilizing component and to plan subsequent robotic motion,
(d) automatic advancement and imaging of the first extension tube toward the oropharynx, followed by image acquisition and analysis to confirm position and plan the next movement,
(e) automatic advancement of the second extension tube toward the epiglottis, with corresponding imaging and trajectory correction,
(f) automatic extension of the third extension tube beyond the epiglottis into the trachea, thereby achieving accurate tracheal entry under continuous visual confirmation,
(g) advancement of the endotracheal tube over the first, second, and third extension tubes, sequentially guiding it into the trachea under robotic control,
(h) retraction sequence of the guiding tubes, wherein the third extension tube retracts into the second, the second retracts into the first, and the first retracts into the external arm, leaving the endotracheal tube securely positioned in the trachea.

By combining high-resolution imaging, machine-vision processing, and precision robotic actuation, the present invention enables automated, adaptive, and safe placement of an endotracheal tube. This represents a substantial improvement over conventional manual and video-assisted techniques, particularly in scenarios involving limited visibility, atypical anatomy, or operator inexperience. The system may be deployed in operating rooms, intensive care units, emergency departments, or prehospital environments, offering a consistent and reproducible approach to airway management.

Moreover, the use of machine learning models allows the apparatus to continuously improve its performance through accumulated procedural data. Over time, the system can refine its recognition algorithms, optimize motion planning for varied anatomies, and further reduce intubation time and complication rates. The invention thus lays the foundation for a new generation of intelligent robotic airway management systems capable of performing one of the most critical procedures in medicine with unprecedented accuracy, safety, and efficiency.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 demonstrates a cross-section of a typical subject anatomy, in particular regions that are of interest in the surgical procedure of an endotracheal intubation.

FIG. 2 demonstrates an external view of the subject's anatomy when holding the mouth open for evaluation of intubation difficulty.

FIG. 3 demonstrates an internal view of the subject's anatomy once the imaging module has entered past the subject's mouth, and the epiglottis can be visualized.

FIG. 4 demonstrates a cross-sectional view of a subject's anatomy with a preferred embodiment of the robotic system placed on the chest of the subject to be treated.

FIG. 5 demonstrates the same subject anatomy as in FIG. 4, with a first extension telescoping from the extension arm.

FIG. 6 demonstrates a closer image of the same subject anatomy as in FIG. 5 with a third extension tube, telescoping from a second extension tube, which in turn is telescoping from the first extension tube.

FIG. 7 demonstrates the same subject anatomy as in FIG. 5, but illustrates the third extension tube's movement into its final position within the subject's anatomy.

FIGS. 8 through 11 show different parts of a block diagram illustrating the steps of one exemplary method of the present invention.

FIGS. 12 through 14 demonstrate successive steps of advancement of different portions of the extension arm from within a preceding part thereof.

FIGS. 15 and 16 demonstrate a change in curvature of the second extension tube.

FIGS. 17 and 18 demonstrate a change in curvature of the third extension tube.

### DETAILED DESCRIPTION OF PREFERRED EMBODIMENTS OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

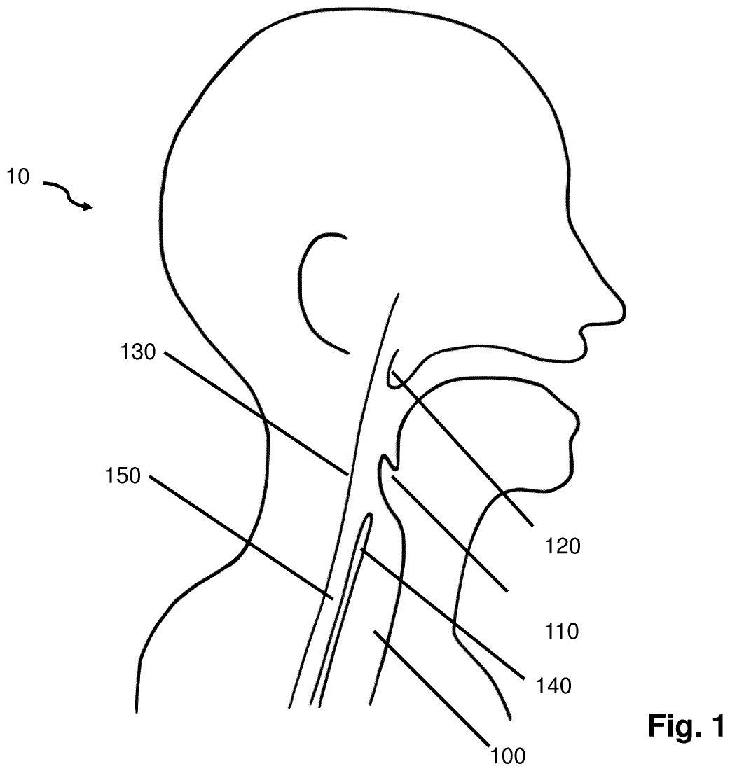

FIG. 1 illustrates a sagittal view of the subject's head and neck anatomy 10, showing the anatomical structures relevant to the endotracheal intubation procedure. The trachea 100 is identified as the target location for the distal end of the system, while the esophagus 150 is shown as an adjacent structure that must be avoided during ventilation. Key anatomical landmarks that assist the machine vision system in guiding the device are labeled, including the epiglottis 110, the uvula 120, the pharyngeal wall 130, and the cricoid cartilage 140. These features serve as critical reference points for image-based localization and motion planning.

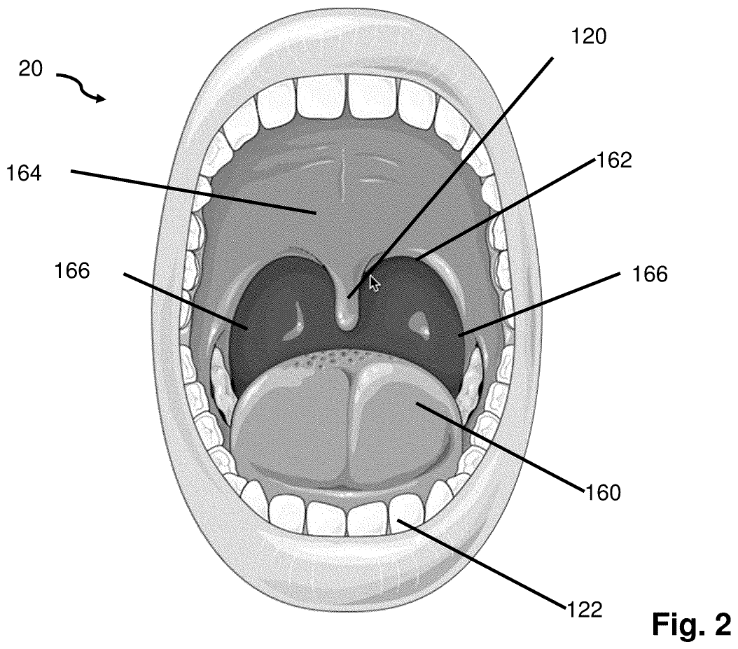

FIG. 2 shows an image capture of the first section of the subject's anatomy, corresponding to the oral cavity 20. Several key features are labeled to support image recognition and machine vision analysis, including the tongue 160, uvula 120, teeth 122, regions of the two palatine tonsils 166, soft palate 162, and hard palate 164. This region serves as the initial environment for image acquisition and system alignment. As the distal end of the device advances beyond the oral cavity into the throat, the robotic mechanism continues to capture and analyze images of additional anatomical structures to guide its motion. Upon positioning near the epiglottis 110, the distal end or a concentrically nested distal section of smaller diameter may articulate and advance at a controlled angle to enter the trachea 100 through the opening formed by the epiglottis.

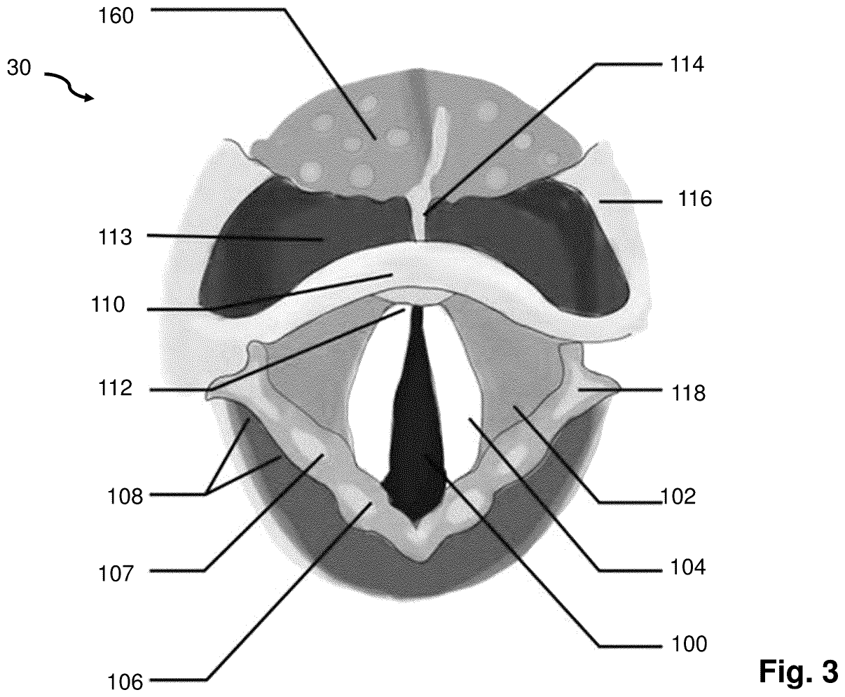

FIG. 3 illustrates an image capture of a second anatomical region, the oropharynx 30. This region presents a rich array of anatomical features that serve as predictive reference points for the machine vision algorithms. Prominent structures include the tongue 160, epiglottis 110, and trachea 100. Additional details in the vicinity of the epiglottis 110 include the tubercle of the epiglottis 112, vallecula 113, median glossoepiglottic fold 114, lateral glossoepiglottic fold 116, and aryepiglottic fold 118. In the area surrounding the entrance to the trachea 100, further features are visible, such as the ventricular fold 102, vocal fold 104, corniculate cartilage 106, cuneiform cartilage 107, and piriform recess 108. These detailed anatomical features are integral to the system's training data and play an essential role in the image-based prediction and navigation algorithms used for accurate and safe endotracheal placement.

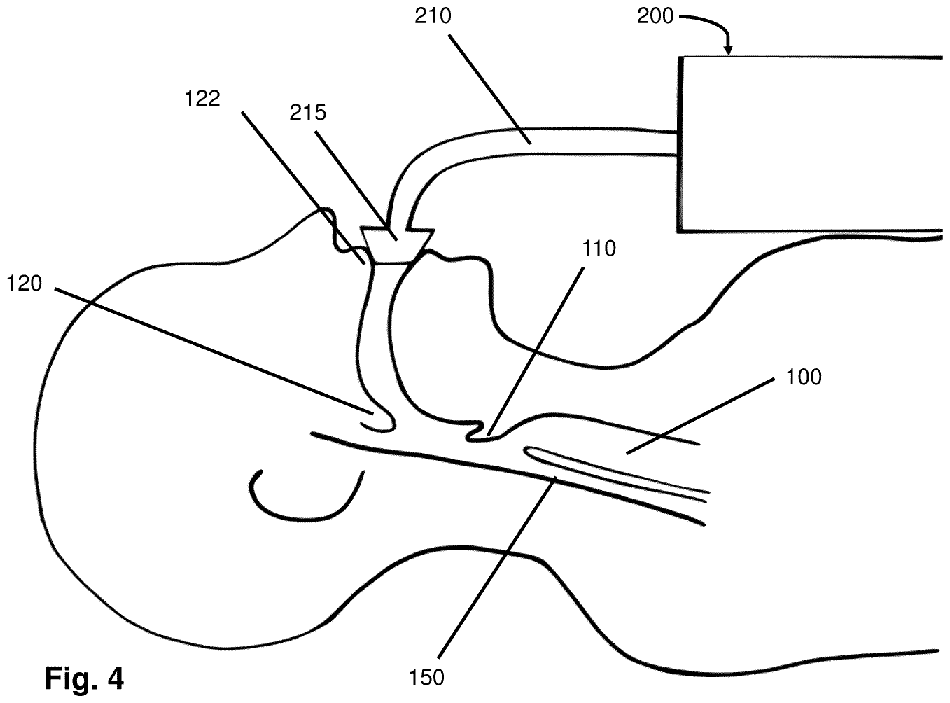

In broad terms, the apparatus for machine-vision guided endotracheal intubation may include a robotic component 200 and a controller. The robotic component 200, in turn, may include a guiding tube robotic mechanism and an actuation module. The guiding tube robotic mechanism may include an external arm 210 and a housing 200 enclosing the actuation module and, optionally, the controller, as seen in FIG. 4.

In embodiments, the robotic guiding mechanism of the present invention comprises an external arm configured to serve as both a structural support and an initial positioning interface for the system. A First stabilizing component 215, such as a teeth bite block, may be positioned at the distal end of the external arm 210. The external arm 210 may be mounted on, or articulated by, the actuation module and is configured to enter the subject's oral cavity under controlled motion. The external arm 210 provides a stable base through which a plurality of telescoping extension tubes are deployed.

The external arm 210 may house within its internal lumen a first extension tube 220, which in turn may contain a second extension tube 230, and a third extension tube 232 positioned within the second. The first, second, and third extension tubes 220, 230, and 232 may be configured to extend telescopically and, at least in some embodiments, concentrically along a common longitudinal axis, thereby forming a multi-stage deployment system. This telescopic design allows the apparatus to advance distally in a controlled, incremental manner through the oral cavity, past the oropharynx, and into the trachea.

Each of the extension tubes may be configured to extend away from the external arm 210 to a combined overall length sufficient to reach a desired position within the subject's trachea. The advancement of each extension tube may be individually or sequentially controlled by the actuation module, which is in turn directed by the controller executing machine-vision-based motion planning algorithms.

The extension tubes may be fabricated from flexible, biocompatible materials such as polyurethane, silicone, Pebax®, or thermoplastic elastomers. Wall thickness and material selection are optimized to achieve a balance between structural integrity and flexibility. This allows the extension tubes to bend and conform to the anatomical contours of the oral cavity, pharynx, and trachea, while maintaining sufficient stiffness to transmit pushing and rotational forces during insertion.

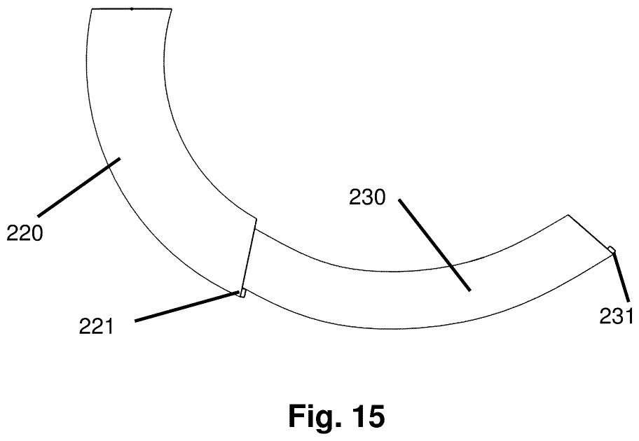

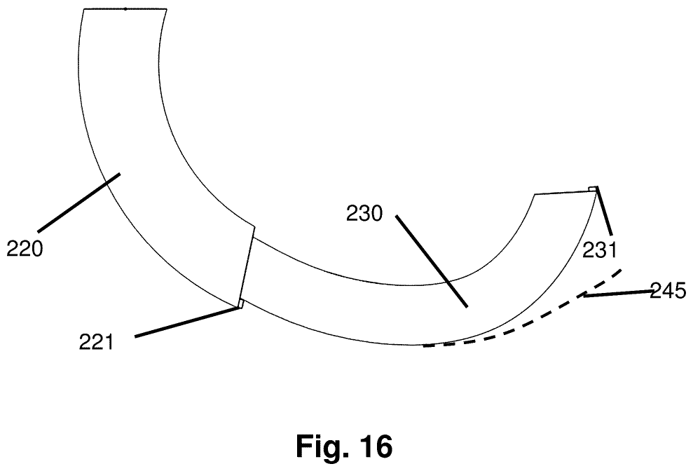

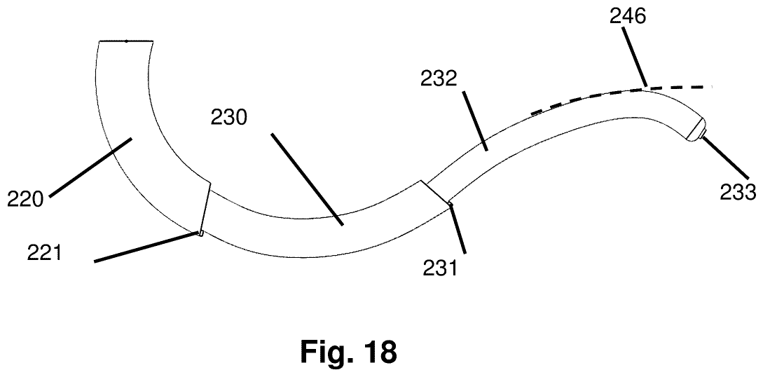

Curvature control of the extension tubes may be accomplished through the incorporation of one or more pull wires embedded within or along the walls of the tubes—see FIGS. 15 through 18. Each pull wire may extend longitudinally along the length of the tube and may terminate at or near the distal end thereof. The actuation module may selectively apply tension to one or more of these pull wires to induce a controlled bending motion, thereby enabling directional steering of the distal tip from the original position 245 and 246. This allows the robotic mechanism to dynamically adjust curvature and trajectory in response to anatomical variations, obstructions, or real-time imaging feedback.

The tension in the pull wires may be regulated by servo-driven actuators or micro-motors within the actuation module. The controller interprets the imaging data acquired by the system and issues corresponding control signals to adjust the curvature and extension rate of each tube segment, ensuring safe and accurate navigation toward the tracheal opening.

The overall external diameter of the assembly—comprising the external arm and the nested extension tubes—may be selected to permit the sliding of a standard endotracheal tube over the external arm or, at least, over the first extension tube 220. For adult applications, where typical endotracheal tubes have an internal diameter ranging from approximately 7 mm to 12 mm, the first extension tube 220 may be dimensioned to slidingly fit within the lumen of the endotracheal tube while still allowing smooth advancement and retraction. For pediatric or neonatal applications, the diameter of the first extension tube 220 may be reduced proportionally, and the overall number of telescoping stages may be modified.

In certain embodiments, the robotic mechanism may include only two extension tubes (e.g., 220 and 230) to simplify design and reduce size. In other embodiments, particularly those adapted for adult or complex airway cases, the mechanism may include four or even five telescoping tubes to increase reach and maneuverability. The number of telescoping elements may therefore vary according to the target subject population, clinical use case, or specific anatomical requirements, and the invention is not limited in this regard.

The walls of the first, second, and third extension tubes may further incorporate electrical conductors or flexible printed circuits (FPCs) that provide power and signal transmission to one or more image capture modules positioned at the distal ends of the respective tubes. In one embodiment, a first imaging module may be positioned at the distal tip of the external arm 210 to capture images of the oral cavity; a second imaging module may be located at the distal end of the first extension tube 220 to visualize the oropharynx; and a third imaging module may be provided at the distal end of the second or third extension tube to image the epiglottis, vocal cords, or tracheal lumen.

Each image capture module may include at least one miniature video camera, such as a CMOS or CCD sensor, coupled with an integrated light source, such as one or more light-emitting diodes (LEDs) or fiber-optic illuminators, to provide sufficient illumination of the airway structures. The imaging data from each module may be transmitted to the controller in real time, where it is analyzed using machine-vision algorithms to identify anatomical features and guide subsequent robotic motion.

In some embodiments, each imaging module may also include depth-sensing or stereoscopic capability to facilitate three-dimensional mapping of the airway and to enhance spatial awareness during navigation. The combination of sequential telescopic extension, controlled curvature, and multi-point imaging enables the robotic system to advance toward the trachea in a stepwise and adaptive fashion, ensuring continuous visual confirmation of anatomical landmarks and minimizing the risk of esophageal misplacement or tissue injury.

In one embodiment, the actuation module forms the central mechanical and electromechanical interface responsible for advancing, retracting, and articulating the extension tubes of the robotic component. The actuation module may be mounted to a fixed base or support frame located outside the subject's oral cavity and may include a plurality of linear and rotary actuators, servomotors, and drive assemblies configured to impart controlled motion to the external arm and the telescoping extension tubes.

The actuation module may further include motorized drive systems coupled to the proximal ends of the extension tubes. Each drive system may employ a lead-screw, rack-and-pinion, cable-driven, or pneumatic actuator configured to advance or retract its corresponding tube segment along the longitudinal axis. The relative positions of the tubes may be continuously monitored using position sensors such as encoders, optical displacement sensors, or linear potentiometers. These sensors provide real-time feedback to the controller to enable closed-loop motion control and ensure that each tube segment moves in coordination with the others.

In some embodiments, each telescoping tube may be coupled to a dedicated drive channel within the actuation module. The drive channels may be independently or sequentially activated to produce stepwise extension of the first, second, and third tubes. This arrangement allows fine control of tube advancement and retraction during various stages of intubation and may facilitate smooth transitions between imaging, navigation, and tube placement operations.

In addition to linear motion control, the actuation module may include one or more curvature adjustment systems for steering the distal ends of the extension tubes. These systems may consist of servomotor-driven spools or pulleys configured to apply controlled tension to the pull wires embedded within the walls of the tubes. The actuation of the pull wires may be distributed across multiple channels to allow precise adjustment of bending angles and directional orientation in multiple planes. By modulating the amount and direction of pull wire tension, the controller can dynamically shape the curvature of the distal segments to guide the robotic assembly along a safe and anatomically optimized trajectory.

In some embodiments, the actuation module may include force or pressure sensors integrated at the interface between the drive system and each tube. These sensors may detect abnormal resistance or excessive force encountered during advancement, providing an additional safety layer that prevents tissue trauma or device misalignment. Upon detection of excessive force, the controller may automatically halt or retract the advancing tube and initiate a corrective trajectory plan based on updated imaging feedback.

The controller, in communication with the actuation module, serves as the system's computational and decision-making center. The controller may include one or more microprocessors, graphics processing units (GPUs), or dedicated AI accelerators capable of performing high-speed image analysis and real-time motion control. The controller may execute a plurality of software modules responsible for image acquisition, feature recognition, trajectory planning, actuation control, and safety management.

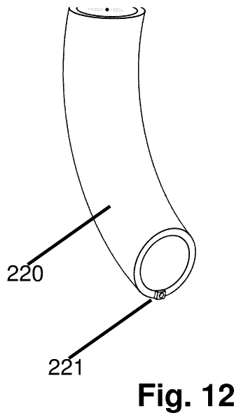

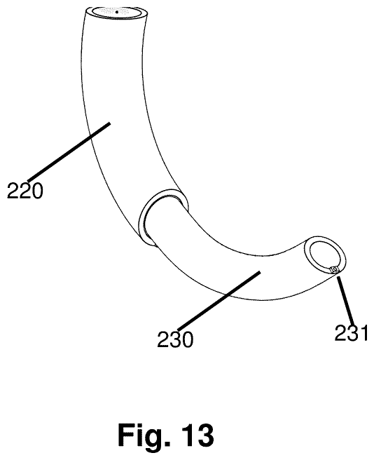

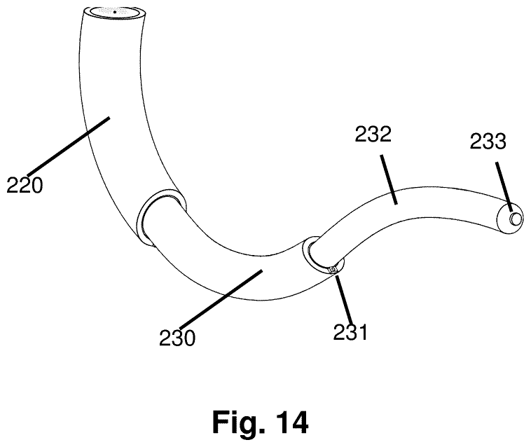

The machine-vision subsystem of the controller may receive continuous image streams from one or more imaging modules positioned at the distal ends of the external arm and the telescoping extension tubes. These image capture modules 221, 231, 233 may each include a miniaturized video camera and one or more illumination sources, allowing for continuous visualization of the internal anatomy from multiple perspectives—see FIGS. 12 through 14. The received image data may be processed in real time by a computer vision element forming an integral part of the software component of the system.

The primary function of the computer vision element is to determine, from the captured visual images, the anatomical location of the robotic distal tip within the patient's airway. This may be accomplished through the use of advanced artificial intelligence (AI) algorithms trained to recognize key airway landmarks, such as the tongue, uvula, soft palate, epiglottis, vocal cords, and tracheal rings. Feature recognition and segmentation may be performed using convolutional neural networks (CNNs) or similar deep learning architectures specifically adapted for endoscopic and airway imaging.

The performance of the machine-vision and control subsystems may be enhanced through the use of prior data collected from recorded intubation procedures, such as the data recorded from video laryngoscopes. During system development and calibration, image and motion data may be gathered from a large number of human or mannequin-based intubations performed under various anatomical conditions and lighting environments. Each recorded dataset may include synchronized video streams from one or more onboard cameras, positional feedback from the robotic actuators, force or pressure sensor data (if available), and corresponding procedural outcomes such as successful or unsuccessful placement.

The collected data may be used to train machine-learning models employed by the computer vision element. Specifically, convolutional neural networks (CNNs) or transformer-based vision architectures may be trained to automatically identify and segment relevant anatomical structures, such as the tongue, epiglottis, glottic opening, and tracheal rings, under different patient anatomies, lighting conditions, and orientations. The datasets may include a variety of subjects, airway geometries, and procedural contexts, enabling the system to generalize across a wide population.

In certain embodiments, supervised learning techniques may be utilized, wherein expert clinicians annotate the recorded images to indicate the precise location of anatomical landmarks, optimal tool paths, and instances of correct versus incorrect tube placement. These labeled datasets provide ground truth for training and validation of the AI algorithms, allowing the system to improve recognition accuracy and robustness.

In other embodiments, reinforcement learning or imitation learning frameworks may be employed. In such approaches, the robotic controller may learn optimal motion trajectories and actuation strategies by observing previously executed intubation sequences —either performed manually by clinicians or autonomously by earlier versions of the system. Through iterative training cycles, the system may learn to associate visual inputs with successful motion commands, optimizing for criteria such as minimal tissue contact, shortest path to tracheal entry, and highest procedural success rate.

The system's learning process may be continuous, such that each new intubation contributes additional data to the training set. Over time, the AI algorithms may be refined through periodic retraining on the expanded dataset, further enhancing their ability to recognize anatomical landmarks and predict safe, efficient trajectories. In certain embodiments, the controller may also employ online learning, enabling it to adapt its performance to subtle patient-specific variations in real time.

In addition to image-based training, motion and force data from previous procedures may be used to construct a kinematic and dynamic model of the robotic insertion process. This model may assist the controller in predicting the mechanical response of the extension tubes and external arm to applied actuator inputs and anatomical resistance, thereby improving motion precision and stability.

Through the integration of these recorded datasets and adaptive learning methodologies, the overall system may achieve progressively higher levels of autonomy, reliability, and procedural safety in both clinical and emergency intubation scenarios.

Based on the machine-recognized features and their spatial relationships, the controller may compute the three-dimensional position and orientation of the robotic distal tip relative to these anatomical landmarks. This information is used by the motion planning subsystem to determine the next advancement vector, orientation, and curvature adjustments needed to safely navigate toward the trachea while minimizing contact with surrounding tissues.

The computer vision system may continuously update this positional information as new image data is received, enabling real-time adaptive control of the robot's motion. The software control element thus integrates both visual feedback and motion commands: it determines which robotic components must be actuated, in what sequence, and with which parameters, in order to achieve the next step in the intubation process.

At any time during robotic advancement, the vision subsystem remains active, continuously analyzing incoming images to reassess the robot's location and refine motion trajectories. If the visual analysis indicates successful passage through the vocal cords and correct placement within the trachea, the controller may autonomously transition to the next procedural steps—such as inflating the tracheal cuff and activating the ventilation apparatus to initiate airflow to the lungs.

In some embodiments, the machine vision element may also incorporate depth mapping, optical flow analysis, or stereoscopic imaging to improve spatial localization and depth perception in complex airway geometries. The combination of continuous visual input, AI-based anatomical recognition, and adaptive motion planning provides a closed-loop control system that enhances both the precision and safety of automated or semi-automated intubation procedures.

The controller may also manage synchronization between imaging and actuation cycles, ensuring that each incremental movement is followed by image capture and verification before further advancement occurs. This cyclical control structure provides continuous visual confirmation and precise localization of the distal end of the robotic mechanism within the airway.

In some embodiments, the controller may include or communicate with a user interface module, which may display real-time video streams, reconstructed three-dimensional airway maps, and system status information. The user interface may allow an operator, such as an anesthesiologist, respiratory therapist, or emergency physician, to monitor the automated intubation process, manually override specific motions if needed, and confirm final placement of the endotracheal tube.

Furthermore, the controller may include a data acquisition and learning subsystem configured to record procedural parameters, imaging data, and system responses. This data may be used to refine and retrain the underlying machine-learning models, improving feature recognition accuracy and motion planning performance over time. As the system accumulates procedural experience across diverse patient populations and anatomical variations, its predictive accuracy and safety performance may progressively increase.

In certain embodiments, the controller may be configured to interface with external hospital systems, such as anesthesia monitoring platforms or electronic medical record (EMR) databases, to receive patient-specific information (e.g., age, height, weight, or known airway characteristics). Such integration allows the system to automatically select optimal operating parameters and intubation profiles tailored to the individual subject, further enhancing procedural safety and efficiency.

Integration of the actuation module with the intelligent controller enables closed-loop robotic control of endotracheal intubation. The system can autonomously interpret the visual scene, plan and execute precise tube advancement motions, detect and correct deviations, and confirm successful tracheal placement. The combination of high-resolution imaging, adaptive robotic mechanics, and machine-learning-driven feedback control provides a substantial improvement over conventional manual and video-assisted airway management techniques, reducing operator dependency and minimizing the risk of adverse outcomes.

The present invention further provides a method of machine-vision guided endotracheal intubation, performed by a robotic system incorporating the guiding tube mechanism, actuation module, and machine-vision controller as described herein. The method enables safe, precise, and semi-or fully-autonomous placement of an endotracheal tube into a subject's trachea.

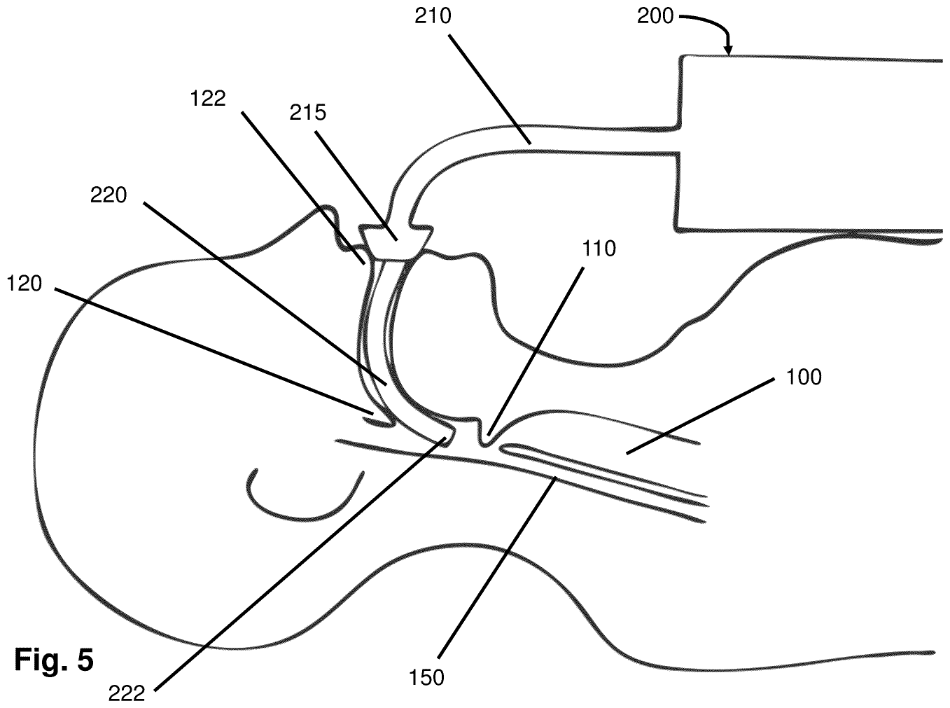

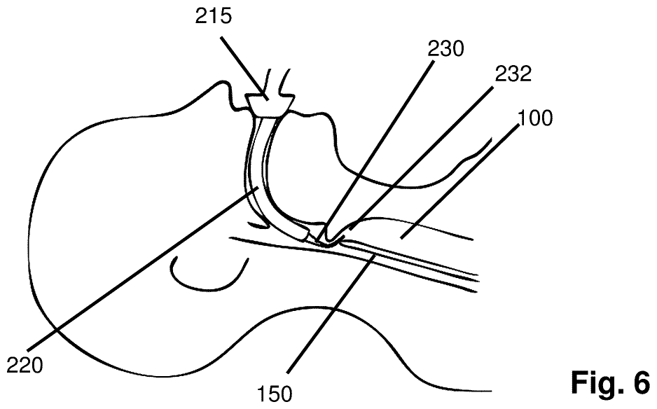

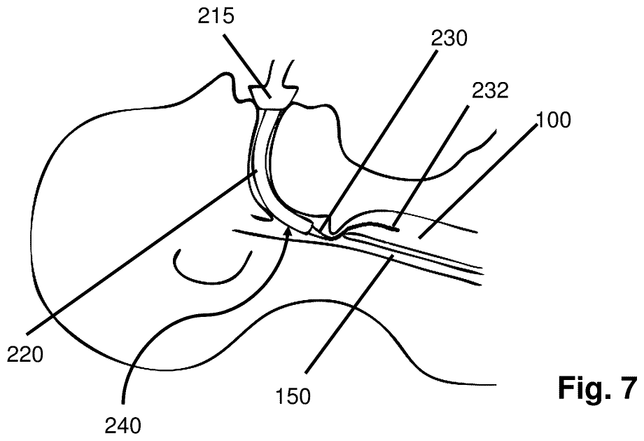

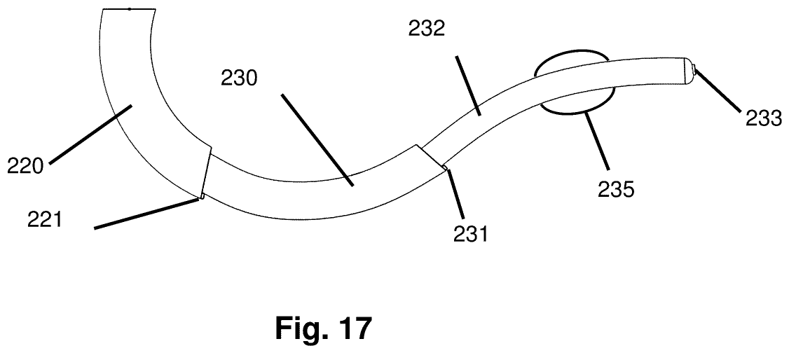

The method may comprise the following steps:
(a) positioning of a conventional endotracheal tube over a first extension tube of a guiding tube robotic mechanism.
(b) positioning the first stabilizing component of the external arm adjacent to a first positional marker in the oral cavity of the subject—see FIG. 4. This establishes a fixed and repeatable reference point from which subsequent robotic motions may be planned and executed.
(c) operating the controller to automatically take and process an image of the oral cavity to determine the position of the first stabilizing component 215 and to plan robotic motion therefrom. The machine-vision subsystem may analyze the captured image, identify relevant anatomical landmarks such as the teeth, tongue, and soft palate, and calculate the optimal orientation for initial insertion.
(d) operating the controller to automatically actuate the guiding tube robotic mechanism to advance the first extension tube 220 from the external arm 210 to a position adjacent to the oropharynx—see FIG. 5. The advancement may occur under continuous visual feedback, with incremental image acquisition and motion correction to ensure safe navigation within the oral cavity.
(e) operating the controller to automatically take and process an image of the oropharynx to determine the position of the first extension tube 220 and to plan further robotic motion therefrom. The image analysis may include identification of the uvula, soft palate, and posterior pharyngeal wall to guide the next movement.
(f) operating the controller to automatically actuate the guiding tube robotic mechanism to advance the second extension tube 230 from the first extension tube 220 to a position adjacent to the epiglottis.
(g) operating the controller to automatically take and process an image of the epiglottis to determine the position of the second extension tube 232 and to plan robotic motion therefrom. The machine-vision system identifies anatomical features, such as the glottic opening and vocal cords, as visual cues for tracheal entry.
(h) operating the controller to automatically actuate the guiding tube robotic mechanism to advance the third extension tube 232 from the second extension tube 230 to a position within the trachea—see FIGS. 6 and 7—preferably, without touching the epiglottis. During this step, the system may employ curvature adjustments and trajectory optimization to minimize tissue contact and to achieve precise tracheal alignment. An optional balloon 235 (seen in FIG. 17) at the distal portion of the third extension tube 232 may be inflated to secure its position in the trachea.
(i) advancing the endotracheal tube over the first, second, and third extension tubes, in sequence, until the distal end of the endotracheal tube reaches a suitable position within the trachea. In one embodiment, the endotracheal tube may slide over the telescoping assembly, collectively referred to as the actuated bougie 240, which serves as a guiding rail for smooth placement.
(j) operating the controller to automatically retract the third extension tube into the second extension tube, followed by retraction of the second extension tube into the first extension tube, and subsequently retracting the first extension tube into the external arm, thereby completely removing the guiding tube robotic mechanism while maintaining the endotracheal tube in place.

In embodiments, at least one, two, or all three steps (d), (f), or (h) may further comprise adjusting the curvature of the respective first, second, or third extension tube. Such curvature adjustment may be achieved by tensioning pull wires integrated within the tube walls and actuated by the control module. Controlled bending of each segment allows navigation through variable airway geometries and avoidance of anatomical obstructions.

Furthermore, steps (c), (e), and (g) may each include a substep of identifying suitable anatomical features from the images available to the controller. These features may include the tongue, oropharyngeal arch, epiglottis, vocal cords, or tracheal rings. The machine-vision subsystem employs trained neural networks or similar pattern recognition algorithms to detect and classify such structures, enabling the controller to dynamically plan the next robotic action.

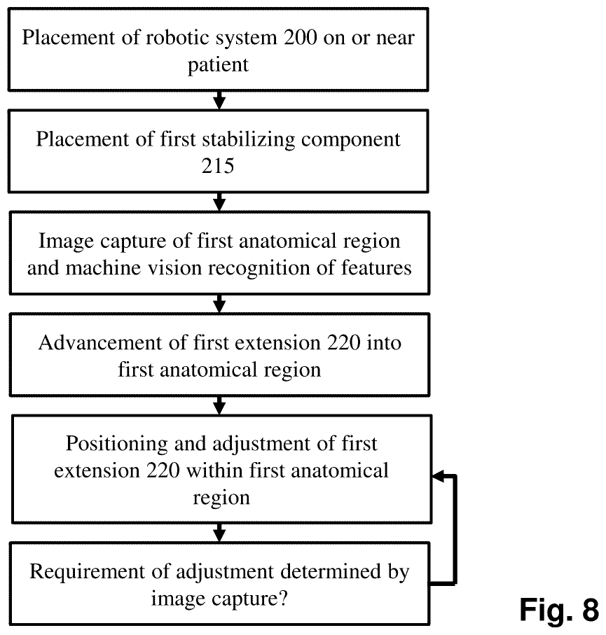

FIGS. 8 through 11 illustrate an exemplary embodiment of the intubation method as performed by the robotic system 200. In FIG. 8, the robotic system 200 is positioned near the subject, and the first stabilizing component 215 of the external arm 210 is placed within the oral cavity 20. The imaging module 221 located at the distal end of the first extension tube 220 captures the first set of images of the first anatomical region, the oral cavity. The machine-vision subsystem processes these images to recognize anatomical features and to plan the controlled advancement of the first extension tube 220 through the oral cavity toward the oropharynx. Additional image acquisition and adjustment cycles may occur until the distal end of the first extension tube 220 is correctly positioned.

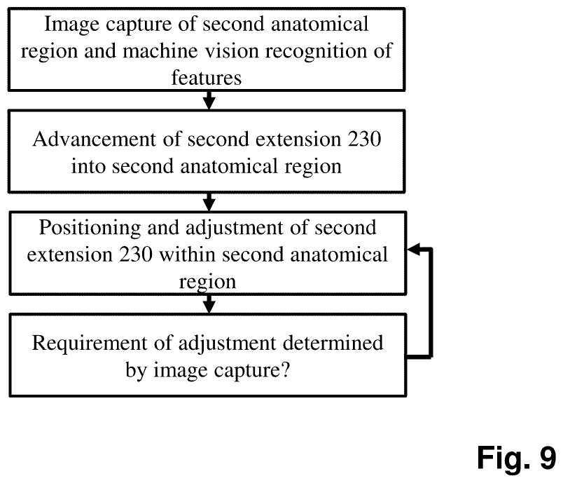

In FIG. 9, the process continues as the second extension tube 230 is deployed from within the first extension tube 220. The imaging module 231 at the distal tip of the second extension tube 230 captures views of the second anatomical region, the pharyngeal region. The controller interprets these images to identify the soft palate, posterior wall, and emerging epiglottis. Based on these inputs, the controller plans and executes the advancement of the second extension tube toward the epiglottic region, continuously refining motion trajectories through real-time feedback.

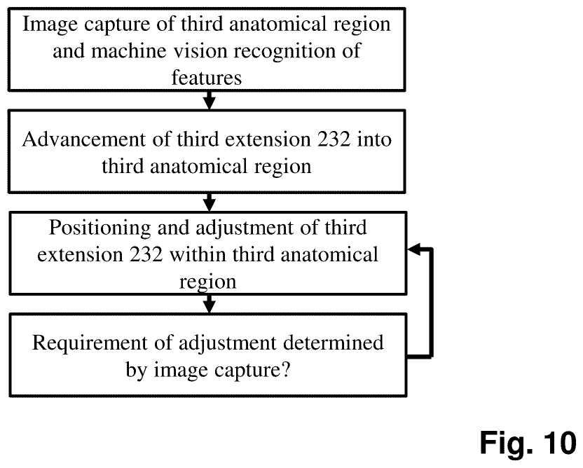

In FIG. 10, the third extension tube 232 is advanced distally from the second extension tube 230 toward and into the trachea 100. The imaging module provides real-time visualization of the glottic opening, vocal cords, and tracheal rings. Using these images, the controller confirms correct tracheal entry and stabilizes the assembly for subsequent placement of the endotracheal tube.

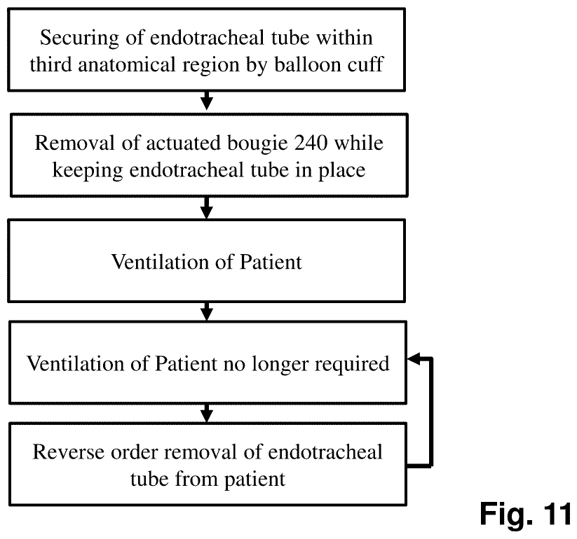

In FIG. 11, after successful tracheal placement, the endotracheal tube is advanced over the actuated bougie 240, comprising all three deployed extension tubes, until its distal end reaches the desired position. Once positioned, the guiding mechanism is retracted in reverse order, leaving the endotracheal tube in place for ventilation. The system may then automatically proceed with secondary tasks such as tracheal cuff inflation and initiation of controlled ventilation through an associated ventilation apparatus.

Following the completion of ventilation or upon clinical determination that intubation is no longer required, the endotracheal tube may be withdrawn manually or with robotic assistance. In some embodiments, reinsertion of the actuated bougie 240 may facilitate controlled extraction of the tube and safe removal of all robotic components.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Incorporation by reference is limited such that no subject matter is incorporated that is contrary to the explicit disclosure herein, no claims included in the documents are incorporated by reference herein, and any definitions provided in the documents are not incorporated by reference herein unless expressly included herein.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. Apparatus for machine-vision guided endotracheal intubation, the apparatus comprising:
a robotic component, in turn, comprising a guiding tube robotic mechanism and an actuation module, wherein the guiding tube robotic mechanism comprises:
an external arm terminating with a first stabilizing component and configured to capture an image of an oral cavity,
a first extension tube positioned inside the external arm and configured to extend distally therefrom when advanced by the actuation module, the first extension tube defining a first distal end thereof configured to capture an image of an oropharynx,
a second extension tube positioned inside the first extension tube and configured to extend distally from the distal end thereof when advanced by the actuation module, and
a third extension tube positioned inside the second extension tube and configured to extend distally therefrom when advanced by the actuation module,
a controller configured to operate the robotic component based on processed machine-vision tasks using captured images of the oral cavity, the oropharynx, and the epiglottis.

2. The apparatus, as in claim 1, wherein the machine-vision tasks to be processed by the controller comprise image recognition, robotic motion planning, and actuation of the guiding tube robotic mechanism.

3. The apparatus, as in claim 1, wherein at least one of the first extension tube, the second extension tube, or the third extension tube is configured to change a curvature in response to the actuation module.

4. The apparatus, as in claim 3, wherein at least one of the first extension tube, the second extension tube, or the third extension tube comprises actuatable elements configured to change the curvature of the respective first extension tube, the second extension tube, or the third extension tube when activated by the actuation module.

5. The apparatus, as in claim 1, wherein the third extension tube comprises an external inflatable balloon.

6. The apparatus, as in claim 1, wherein at least one of the external arm, the first extension tube, the second extension tube, or the third extension tube is equipped with a camera operatively connected to the controller and configured to capture an image in front of the respective external arm, the first extension tube, the second extension tube, or the third extension tube.

7. The apparatus, as in claim 2, wherein the controller is configured to process machine-vision tasks based on predictive machine learning techniques.

8. The apparatus, as in claim 2, wherein the controller is trained using a neural network on a database of prior patient interventions containing laryngoscope images.

9. The apparatus, as in claim 8, wherein the controller is trained to detect anatomical features to perform robotic motion planning and actuation of the guiding tube robotic mechanism.

10. The apparatus, as in claim 9, wherein the controller is configured to process the oral cavity image to recognize at least one of a tongue, a uvula, teeth, two palatine tonsils if present, a soft palate, and a hard palate.

11. The apparatus, as in claim 9, wherein the controller is configured to process the image of oropharynx to recognize at least one of the tongue, an epiglottis, and a trachea.

12. The apparatus, as in claim 11, wherein the controller is configured to process the image of oropharynx to further recognize at least one of a tubercle of the epiglottis, a vallecula, a median glossoepiglottic fold, a lateral glossoepiglottic fold, an aryepiglottic fold, a ventricular fold, a vocal fold, a corniculate cartilage, a cuneiform cartilage, and a piriform recess.

13. The apparatus, as in claim 11, wherein the controller is configured to actuate the advancement of the third extension tube into the trachea without touching the epiglottis.

14. The apparatus, as in claim 1, wherein the controller is configured to actuate the guiding tube robotic mechanism to advance the first extension tube from the external arm, followed by advancing the second extension tube from the first extension tube, followed by advancing the third extension tube from the second extension tube.

15. The apparatus, as in claim 1, wherein the controller is configured, upon completion of endotracheal intubation, to first retract the third extension tube into the second extension tube, followed by retraction of the second extension tube into the first extension tube, followed by retraction of the first extension tube into the external arm, thereby removing entirely the guiding tube robotic mechanism.

16. A method of machine-vision guided endotracheal intubation comprising the following steps:
(a) positioning an endotracheal tube over a first extension tube of a guiding tube robotic mechanism, which in turn comprises:
i. an external arm terminating with a first stabilizing component and configured to capture an image of an oral cavity,
ii. a first extension tube positioned inside the external arm and configured to extend distally therefrom when advanced by an actuation module, the first extension tube defining a first distal end thereof configured to capture an image of an oropharynx,
iii. a second extension tube positioned inside the first extension tube and configured to extend distally from the distal end thereof when advanced by the actuation module, and
iv. a third extension tube positioned inside the second extension tube and configured to extend distally therefrom when advanced by the actuation module,
(b) positioning the first stabilizing component of the external arm adjacent to a first positional marker in an oral cavity of a subject,
(c) operating a controller to automatically take and process an image of the oral cavity to determine a position of the first stabilizing component in the oral cavity and to plan robotic motion therefrom,
(d) operating the controller to automatically actuate the guiding tube robotic mechanism to advance the first extension tube from the external arm to a position adjacent to the oropharynx,
(e) operating the controller to automatically take and process an image of the oropharynx to determine the position of the first extension tube and to plan robotic motion therefrom,
(f) operating the controller to automatically actuate the guiding tube robotic mechanism to advance the second extension tube from the first extension tube to a position adjacent to the epiglottis,
(g) operating the controller to automatically take and process an image of the epiglottis to determine the position of the second extension tube and to plan robotic motion therefrom,
(h) operating the controller to automatically actuate the guiding tube robotic mechanism to advance the third extension tube from the second extension tube to a position in the trachea,
(i) advancing the endotracheal tube over the first extension arm, then over the second extension arm, and then over the third extension arm to a suitable position in the trachea, and
(j) operating the controller to first automatically retract the third extension tube into the second extension tube, followed by retraction of the second extension tube into the first extension tube, followed by retraction of the first extension tube into the external arm, thereby removing entirely the guiding tube robotic mechanism from the endotracheal tube.

17. The method of machine-vision guided endotracheal intubation, as in claim 16, wherein at least one of steps (d), (f), or (h) is accomplished by adjusting a curvature of the respective first extension tube, the second extension tube, or the third extension tube.

18. The method of machine-vision guided endotracheal intubation, as in claim 16, wherein steps (c), (e), and (g) include a step of identifying a suitable anatomical feature from a respective image available to the controller.
